(19) United States
(12) Patent Application Publication
(10) Pub. No.: US 2026/0041705 A1
(43) Pub. Date: Feb. 12, 2026

(54) TOPICAL COMPOSITION FOR THE TREATMENT OF MUCOSAL LESIONS WITH SHORT GELLING TIME

(71) Applicant: INMEDICAL THERAPEUTICS, S.L., VITORIA-GASTEIZ (ES)

(72) Inventors: Raúl PÉREZ PÉREZ, VITORIA-GASTEIZ (ES); Manuel MUÑOZ MORENTIN, CAMPEZO/KANPEZU (ES); Jose María ALONSO CARNICERO, VITORIA-GASTEIZ (ES); Antoni VILLARÓ MARTI, SANT VICENÇ DE MONTALT (ES); Luis Fernando MARTÍN-DELGADO SANTOS, ALCOBENDAS (ES); Luciano CONDE CONDE, SANT CUGAT DEL VALLÈS (ES)

(21) Appl. No.: 19/102,958

(22) PCT Filed: Jul. 19, 2024
(86) PCT No.: PCT/EP2024/070493

§ 371 Date: Feb. 11, 2025

(30) Foreign Application Priority Data
Jul. 20, 2023 (EP) 23382747.6

(51) Int. Cl. A61K 31/728; A61K 9/00; A61K 31/437; A61K 47/10; A61K 47/38; A61P 1/04

(52) U.S. Cl. CPC A61K 31/728; A61K 9/0034; A61K 9/0053; A61K 31/437; A61K 47/10; A61K 47/38; A61P 1/04

(57) ABSTRACT

It relates to a topical composition comprising a) from 0.25 to 1.5% w/w of a hyaluronic acid or a salt thereof, b) from 0.01 to 25% w/w of one or more adhesive agents, and c) from 0.005 to 0.5% w/w of a non-absorbable antibiotic; to delivery devices comprising it; and to its uses in medicine, in particular, in the treatment and/or prevention of mucosal lesions; in the prevention of postpolypectomy syndrome; as adjuvant therapy to mechanical treatments in gastrointestinal perforations, and as sealant treatment in surgical anastomoses and leaks or fistulas in gastrointestinal tract.

This application claims the priority of the European Patent Application EP23382747.6 filed on 20 Jul. 2023.

### TECHNICAL FIELD

The present invention relates to a topical composition comprising hyaluronic acid, one or more adhesive agents, and a non-absorbable antibiotic in very low amounts, and to delivery devices comprising it. It also relates to its uses in medicine, especially as endoscopic shield to treat and/or prevent wounds caused or associated to therapeutic endoscopy.

### BACKGROUND ART

Endoscopy is a minimally invasive procedure that allows diagnosing conditions inside the gastrointestinal, respiratory or urinary tract, by means of an endoscope which is inserted through a body passageway. Advances in endoscopic medicine have led to the development of therapeutic endoscopy that enables physicians to treat numerous conditions using endoscopic techniques such as the removal of polyps and early tumors.

Endoscopic polypectomy and hot biopsy have become routine electrosurgery procedures for the removal of colonic small or very small polyps in the course of colonoscopy. Other techniques such as endoscopic mucosal resections (EMR) and endoscopic submucosal resections (ESD) allow the removal of larger polyps or early-stage colorectal cancer, thus reducing the need for surgical intervention.

Although these techniques are generally very safe and convenient, in a few cases there are still post-operative complications such as bleeding, transmural thermal injury and perforation.

On the one hand, coagulation syndrome (also known as postpolypectomy electrocoagulation syndrome and transmural burn syndrome) may appear after polypectomy with electrocoagulation and refers to the development of abdominal pain, fever, leukocytosis, and peritoneal inflammation in the absence of frank perforation.

On the other hand, extensive bleeding may cause a surgical procedure to be terminated and might require a transfusion, whereas postoperative perforation is an especially serious adverse event, which generally requires emergency surgery.

Therefore, taking into account the large number of therapeutic endoscopy procedures carried out today, it is imperative trying to avoid such complications regardless of the limited (in percentage) number of cases with complications.

Several approaches for avoiding the post-operative complications after therapeutic endoscopy have been described, including, for example, clipping techniques after colorectal endoscopic resection. However, these procedures require special devices or are quite complex, and besides, the duration of clip persistence remains unknown. Furthermore, polyglycolic acid (PGA) sheets and fibrin glue have been described in the art as endoscopic tissue shields to cover wounds after colorectal endoscopic submucosal dissection (ESD). According to this procedure, several PGA sheets need to be placed on the affected tissue with biopsy forceps. After the whole area is covered, it is sprayed with fibrin glue. However, this method requires a significant amount of time and is thought to be inefficient for covering large mucosal defects.

The patent application WO2016135219 discloses topical compositions as endoscopic shields to treat wounds caused by endoscopy, which comprise hyaluronic acid or a salt thereof, one or more adhesive agents, and a non-absorbable antibiotic in an amount from 1.5 to 2.5 wt % with respect to the total weight of the composition. These compositions are liquid at room temperature and can be administered to the gastrointestinal mucosa by using e.g., a syringe coupled to a catheter, which can be introduced via the endoscope. When the composition comes into contact with the mucosa at body temperature, its viscosity increases and acquires the consistency of a gel. However, in the hands of the present inventors these compositions showed relatively long gelling times (around 130 s) and limited long-term stability.

Therefore, there is a need to develop a topical composition, which reduces or avoids post-operative complications after therapeutic endoscopy, contributes to the wound healing, and overcomes the problems of the prior art.

### SUMMARY OF INVENTION

The inventors have surprisingly found that when in the compositions disclosed in the patent application WO2016135219 the weight percentage of the non-absorbable antibiotic is significantly reduced, a composition can be obtained which shows optimised long term-stability and short gelling times.

As can be seen in the examples, while a composition according to the prior art shows a gelling time around 130 s, the compositions according to the invention show shorter gelling times, in some cases as short as 30 s or less. This is very convenient since the product can adhere to any position of the mucosa without spreading as a liquid and the treatment can start earlier.

Additionally, it has been found that the viscosity of the compositions of the invention at 21° C. is lower in comparison with the compositions of the prior art. This has the advantage that the compositions are more fluid and can be more easily administered through an injection device such a syringe since the force required to sustain the movement of the plunger to expel the content of the syringe (i.e., the dynamic glide force, DGF) is lower. This allows using narrower catheters and smaller syringes.

Further, the composition of the invention has improved stability over time with respect to the prior art formulations containing higher amounts of no-absorbable antibiotic. The findings of the present invention are completely unexpected. A skilled person would have thought that when reducing the amount of non-absorbable antibiotic in the formulation the stability against bacterial degradation of the gel would decrease. However, the contrary has been found. Without being bound to theory it is though that when the non-absorbable antibiotic is present in amounts around 1 wt % or higher, this causes physical instability in the formulation because the non-absorbable antibiotic precipitates. The present inventors have found that a concentration from 0.005 to 0.5% w/w of a non-absorbable antibiotic is enough to have stability against bacterial degradation of the gel and improve the physical stability of the formulation over time by avoiding the precipitation of the non-absorbable antibiotic.

Therefore, a first aspect of the present invention relates to a topical composition, in particular suitable for application on gastrointestinal or vaginal mucosa, which comprises:
a) from 0.25 to 1.5% w/w of a hyaluronic acid or a pharmaceutically or veterinary acceptable salt thereof,
b) from 0.01 to 25% w/w of one or more adhesive agents, and
c) from 0.005 to 0.5% w/w of a non-absorbable antibiotic;
wherein the % w/w percentages are expressed with respect to the total weight of the composition, provided that the sum of the amounts of the components is equal to or less than 100%.

Another aspect of the invention relates to a kit comprising a delivery device and an injection device comprising the topical composition as previously defined, wherein the delivery device is suitable to be coupled to the injection device.

The compositions of the invention have proved to reduce the ulcerated area in an in vivo endoscopic study in rats as shown in the examples. Thus, the composition of the invention is useful for wound healing, and/or for preventing complications derived from such lesions.

Therefore, another aspect of the invention relates to a topical composition as defined above for use in the treatment of mucosal lesions and/or for the prevention of complications derived from mucosal lesions, in particular wherein the mucosa is gastrointestinal or vaginal mucosa. This aspect relates to the use of hyaluronic acid or a pharmaceutically or veterinary acceptable salt thereof and a non-absorbable antibiotic for the manufacture of a topical composition as defined above for topical use in the treatment of mucosal lesions and/or for the prevention of complications derived from mucosal lesions. It may also be formulated as a method for the treatment of mucosal lesions and/or for the prevention of complications derived from mucosal lesions in a patient in need thereof, comprising topically applying a therapeutically effective amount of the previously defined topical composition to a subject in need thereof, including a human.

Additionally, the composition of the invention is also useful as sealant treatment in surgical anastomoses in the gastrointestinal tract, such us intestinal anastomoses, which is a surgical procedure to establish communication and restore intestinal continuity between two formerly distant portions of the intestine, after removal of a pathological condition affecting the bowel. It is also useful as sealant treatment in leaks or fistulas in the gastrointestinal tract.

Thus, another aspect of the invention relates to a topical composition as defined above for use as sealant treatment in surgical anastomoses and leaks or fistulas in the gastrointestinal tract. This aspect relates to the use of hyaluronic acid or a pharmaceutically or veterinary acceptable salt thereof and a non-absorbable antibiotic for the manufacture of a topical composition as defined above for topical use a sealant treatment in surgical anastomoses and leaks or fistulas in the gastrointestinal tract. It may also be formulated as a method for the sealant treatment in surgical anastomoses and leaks or fistulas in the gastrointestinal tract in a patient in need thereof comprising topically applying a therapeutically effective amount of the previously defined topical composition to a subject in need thereof including a human.

### BRIEF DESCRIPTION OF DRAWINGS

FIG. 1 shows the viscosity jump with temperature ramp from 21° C. to 37° C. of formulations of Examples 1 and 3 (V: viscosity, ST: step time).

FIG. 2 shows the adhesive test used for measuring the adhesivity of a sample gel (G) at 37° C. to mucin (M).

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply uniformly throughout the specification and claims unless an otherwise expressly set out definition provides a broader definition.

The weight/weight percentages (% w/w) mentioned herein regarding the components of the composition are expressed in weight (e.g., in g) with respect to the total weight of the composition (e.g., in g), provided that the sum of the amounts of the components is equal to 100%.

For the purposes of the present invention, any ranges given include both the lower and the upper endpoints of the range.

The term “about” or “around” as used herein refers to a range of values ±10% of a specified value. For example, the expression “about 10” or “around 10” includes ±10% of 10, i.e., from 9 to 11.

The expression “room temperature” as used herein means a temperature ranging from 20 to 25° C., particularly of about 21° C.

The expression “body temperature” as used herein refers to temperature ranging from 35 to 42° C., particularly from 36 to 38° C., and more particularly of about 37° C.

The present invention relates to a topical composition. For the purposes of the present invention, the term “topical” refers to the local administration of the composition other than systemic (i.e., parenteral and enteral) administration. More particularly, it refers to the application of the composition to body surfaces such as the mucosal membranes, such as oral, gastrointestinal and vaginal mucosa.

As used herein, “viscosity” refers to a measure of the resistance of a fluid to deform under shear stress and describe the fluid's internal resistance to flow and can be measured using a rheometer. The rheological test was performed on a TA Instruments Discovery HR-1 rheometer using a 40 mm parallel plate and a 700 nm gap. Initially, a conditioning experiment was carried out at 21° C. for 600 s. Subsequently, a test was carried out at 21° C. viscosity (flow peak hold) at SR 1 s⁻¹ for 120 s. Finally, the temperature of the peltier was changed to 37° C. and another viscosity test was performed at SR 1 s⁻¹ for 360 s.

The term “adhesion” as used herein refers to the ability of the topical compositions of the invention to bind to the site of topical application or administration, e.g., mucoses, upon contact, by both chemical and physical means, whereby when they are brought into contact work must be done to separate them. The adhesion can be measured as the force required to remove the gelling formulation from a mucin solution using a texture analyser TA.XT Plus. For example, the gel formulation deposited on a cylinder was connected to mucin solution at a rate of 0.5 mm/s and retracted at speed of 1 mm/s at 37° C. The adhesion is measured in mN/s units.

The terms “molecular weight”, “average molecular weight” and “Mw” have the same meaning and are used herein interchangeably. The molecular weight is calculated by the following equation:

$${\overline{M}}_{w} = \frac{{\sum }_{i} {N}_{i} {M}_{i}^{2}}{{\sum }_{i} {N}_{i} {M}_{i}}$$

where Ni is the number of molecules of molecular mass Mi. The mass average molecular mass can be determined by light scattering, size exclusion chromatography (SEC), and sedimentation velocity.

For the purposes of the present invention, the term “thermoreversible” or equivalent expressions thereof such as “thermally reversible” applied to the composition means that it exhibits reverse thermogellation, i.e., it undergoes a change in viscosity when the temperature varies. Thus, the composition is liquid at room temperature and forms a gel at body temperature. The liquid state at room temperature facilitates the administration of the composition to the target mucosa, by using an appropriate injection device, such as for example a syringe or a jet injector, coupled to a delivery device or system, such as a catheter, which can be introduced via an endoscope. When the composition comes into contact with the mucosa at body temperature, its viscosity increases to a higher viscosity state, hence acquiring the consistency of a gel. This has the advantage that the composition remains on the surface of the affected area.

In one embodiment, optionally in combination with one or more features of the various embodiments described above or below, the topical composition is an injectable composition at room temperature which can be administered using an appropriate injection device.

As mentioned above, the topical composition of the invention comprises a hyaluronic acid or a pharmaceutically or veterinary acceptable salt thereof in an amount from 0.25 to 1.5% w/w with respect to the total weight of the composition.

Hyaluronic acid (HA) is a naturally occurring anionic non-sulfated glycosaminoglycan distributed widely throughout connective, epithelial, and neural tissues and part of the extracellular matrix. It consists of multiple repeating disaccharide units of N-acetyl-D-glucosamine and D-glucuronic acid. HA plays an important role in tissue repair by its proliferative and immunomodulatory effect inducing tissue repair promoting healing re-epitelisation instead of scaring.

There is no limitation on the type of the hyaluronic acid salt that can be used, provided that they are pharmaceutically or veterinary acceptable when used for therapeutic purposes. The term “pharmaceutically or veterinary acceptable salt”, embraces salts commonly used such as e.g., alkali metal salts. The preparation of hyaluronic acid pharmaceutically acceptable salts can be carried out by methods known in the art.

Hyaluronic acid and its salts may differ in some physical properties, but they are equivalent for the purposes of the present invention.

Non-limiting examples of pharmaceutically or veterinary acceptable salts include inorganic salts such as sodium hyaluronate, magnesium hyaluronate, potassium hyaluronate, zinc hyaluronate, cobalt hyaluronate, and the like, as well as organic salts such as tetrabutylammonium hyaluronate, and the like.

In one embodiment, optionally in combination with one or more features of the various embodiments described above or below, the topical composition comprises a hyaluronic acid pharmaceutically or veterinary acceptable salt, more particularly, hyaluronate sodium.

In another embodiment, optionally in combination with one or more features of the various embodiments described above or below, the topical composition comprises a hyaluronic acid or a pharmaceutically or veterinary acceptable salt thereof, more particularly hyaluronate sodium, in an amount from 0.55 to 1.25% w/w, more particularly about 0.8% w/w, with respect to the total weight of the topical composition.

In another embodiment, optionally in combination with one or more features of the various embodiments described above or below, the topical composition comprises a hyaluronic acid or a pharmaceutically or veterinary acceptable salt thereof, more particularly hyaluronate sodium, in an amount about 0.25% w/w, about 0.3% w/w, about 0.35% w/w, about 0.4% w/w, about 0.45% w/w, about 0.5% w/w, about 0.55% w/w, about 0.6% w/w, about 0.65% w/w, about 0.7% w/w, about 0.75% w/w, about 0.8% w/w, about 0.81% w/w, about 0.82% w/w, about 0.85% w/w, about 0.9% w/w, about 0.95% w/w, about 1% w/w, 1.05% w/w, about 1.1% w/w, about 1.15% w/w, about 1.2% w/w, about 1.25% w/w, about 1.3% w/w, about 1.35% w/w, about 1.4% w/w, about 1.45% w/w, or about 1.5% w/w with respect to the total weight of the topical composition.

In another embodiment, optionally in combination with one or more features of the various embodiments described above or below, the hyaluronic acid or the salt thereof, more particularly hyaluronate sodium, has an average molecular weight (Mw) from 5,000 to 1,000,000 Daltons, more particularly, the average molecular weight (Mw) is about 5,000, about 7,500, about 10,000, about 15,000, about 20,000, about 22,500, about 25,000, about 50,000, about 100,000, about 200,000, about 300,000, about 400,000, about 500,000, about 600,000, about 750,000, about 800,000, about 850,000, or about 900,000 Daltons.

In another embodiment, optionally in combination with one or more features of the various embodiments described above or below, the hyaluronic acid or the salt thereof, more particularly hyaluronate sodium, has an average molecular weight (Mw) from 7,500 to 22,500 Daltons, more particularly from 10,000 to 20,000 Daltons.

In another embodiment, optionally in combination with one or more features of the various described above or below, the hyaluronic acid or the salt thereof has an average molecular weight (Mw) from 400,000 to 800,000 Daltons, more particularly from 500,000 to 750,000 Daltons.

The topical composition of the invention further comprises one or more adhesive agents in an amount from 0.01 to 25% w/w with respect to the total weight of the composition. In one embodiment, optionally in combination with one or more features of the various described above or below, the one or more adhesive agents are present in an amount from 10 to 25% w/w, more particularly from 15 to 25% w/w, even more particularly about 15% w/w, about 16% w/w, about 17% w/w, about 18% w/w, about 19% w/w, about 20% w/w, with respect to the total weight of the composition.

In another embodiment, optionally in combination with one or more features of the various embodiments described above or below, the weight ratio between the one or more adhesive agents and the hyaluronic acid or its salt is from 15:1 to 25:1, more particularly from 20:1 to 25:1, even more particularly is about 20:1, about 20.5:1, about 20,75:1, about 21:1, about 21.5:1, about 22:1, about 23:1, about 24:1, or about 25:1.

Non limiting examples of adhesive agents that can be used include polyvinyl acetate, cellulose derivatives, sodium alginate, starch, dextrin, a polyvinyl alcohol (PVA), a (poly) vinyl resin, sodium silicate, poloxamers, and the like. When the adhesive agent is sodium alginate, a compound containing divalent ions, such as CaCl₂), is preferably present in the composition.

Non limiting examples of cellulose ether include hydroxypropyl methylcellulose (HPMC), methyl cellulose (MC), hydroxyethyl cellulose (HEC), hydroxypropyl cellulose (HPC), carboxymethylcellulose (CMC), carboxy methylhydroxyethyl cellulose, propyl cellulose, ethylcellulose, 3-O-ethylcellulose, hydroxypropyl methylcellulose phthalate, ethyl(hydroxyethyl) cellulose, cellulose sodium glycolate, 6-O-alkylated cellulose, cellulose octanoate sulfate, cellulose lauroate sulfate, cellulose stearate sulfate, and cationic derivatives thereof, 15 6-O-benzylcellulose, 2,3-di-O-methyl-6-O-benzylcellulose, 2,3-di-O-benzylcellulose, 2,3-di-O-benzyl-6-O-methylcellulose, 2,3,6-tri-O-benzylcellulose, hydroxypropyl methylcellulose acetate succinate, or O-2-[2-(2-methoxyethoxy) ethoxy]acetyl cellulose.

Hydroxypropyl methylcellulose (HPMC or hypromellose) is a partly O-methylated and O-(2-hydroxypropylated) cellulose ether derivative. It may have a percentage of methoxy substitution from 18 to 35%, and more particularly between 28 and 30% and a hydroxypropoxy content from 4.0 to 20.0% and more particularly between 7 and 12%. The average molecular weight of the HPMC is measured as a function of its viscosity so that such parameter may vary between 2.4 mPas and 140,000 mPas, more particularly between 4 mPas and 120 mPas, and more particularly between 10 and 20 mPas. Its average molecular weight may range from 10,000 to 1,500,000 Da, more particularly from 12,000 to 100,000 Da and more particularly from 15,000 to 20,000 Da.

Methyl cellulose (MC) is a methyl ester of cellulose. It may have a percentage of methoxy substitution from 20 to 55% and more particularly from 25 to 33%. The average molecular weight of MC may range from 10,000 to 220,000 Daltons and more particularly from 10,000 to 20,000 Da.

Carboxymethyl cellulose (CMC) is a carboxymethyl ester of cellulose. It may have a substitution degree from 0.70 to 0.99 and more particularly from 0.80 to 0.95. The average molecular weight of MC may range from 100,000 to 1,000,000 Daltons and more particularly from 250,000 to 750,000 Daltons.

Poloxamers, also known as pluronic compounds, are nonionic triblock copolymers composed of a central hydrophobic chain of polyoxypropylene (poly(propylene oxide)) (PPO) flanked by two hydrophilic chains of polyoxyethylene (poly(ethylene oxide)) (PEO). These poloxamers can be generically designated as (PEO—PPO-PEO). The polyoxypropylene (PPO) content in the poloxamer may be from 30 to 90 wt %, more particularly from 60 to 85% w/w. The poloxamer average molecular weight may be from 1000 to 15000 Da.

Non-limiting examples of poloxamers include poloxamer 407 (Pluronic® F-127) and poloxamer 188 (Pluronic® F-68). Poloxamer 188 has an average molecular weight from 7,000 to 10,000 Da and contains from 75 to 85% w/w of ethylene oxide (EO). Poloxamer 407 has an average molecular weight from 9,000 to 15,000 Da and contains from 70 to 75% w/w of ethylene oxide (EO).

According to one embodiment, optionally in combination with one or more features of the various embodiments described above or below, the adhesive agents are thermoreversible adhesive agents.

In another embodiment, optionally in combination with one or more features of the various embodiments described above or below, the one or more adhesive agents are selected from the group consisting of polyvinyl acetate, a cellulose ether, sodium alginate, starch, dextrin, a polyvinyl alcohol (PVA), sodium silicate, poloxamers.

In another embodiment, optionally in combination with one or more features of the various embodiments described above or below, the adhesive agent is alginate and the composition further comprises CaCl₂).

In another embodiment, optionally in combination with one or more features of the various embodiments described above or below, the one or more adhesive agents are selected from the group consisting of a cellulose ether, a poloxamer, and a combination thereof.

In one embodiment, optionally in combination with one or more features of the various embodiments described above or below, the cellulose ether is selected from the group consisting of hydroxypropyl methylcellulose, methyl cellulose, hydroxyethyl cellulose, hydroxypropyl cellulose, carboxy methylhydroxyethyl cellulose, carboxymethylcellulose, propyl cellulose, ethylcellulose, 3-O-ethylcellulose, hydroxypropyl methylcellulose phthalate, ethyl(hydroxyethyl) cellulose, cellulose sodium glycolate, 6-O-alkylated cellulose, cellulose octanoate sulfate, cellulose lauroate sulfate, cellulose stearate sulfate, and cationic derivatives thereof, 15 6-O-benzylcellulose, 2,3-di-O-methyl-6-O-benzylcellulose, 2,3-di-O-benzylcellulose, 2,3-di-O-benzyl-6-O-methylcellulose, 2,3,6-tri-O-benzylcellulose, hydroxypropyl methylcellulose acetate succinate, O-2-[2-(2-methoxyethoxy) ethoxy]acetyl cellulose. More particularly the cellulose ether is present in an amount from 0.01 to 5% w/w, more particularly from 0.5 to 2% w/w, with respect to the total weight of the composition. In another embodiment, the cellulose ether is present in an amount about 0.01% w/w, about 0.05% w/w, about 0.1% w/w, about 0.2% w/w, about 0.3% w/w, about 0.4% w/w, about 0.5% w/w, about 0.6% w/w, about 0.62% w/w, about 0.7% w/w, about 0.75% w/w, about 0.8% w/w, about 0.9% w/w, about 1% w/w, about 1.1% w/w, about 1.2% w/w, about 1.22% w/w, about 1.3% w/w, about 1.4% w/w about 1.5% w/w, about 1.6% w/w, about 1.62% w/w, about 1.63% w/w, about 1.7% w/w, about 1.8% w/w, about 1.9% w/w, about 2% w/w, about 2.5% w/w, about 3% w/w, about 3.5% w/w, about 4% w/w, about 4.5% w/w, or about 5% w/w, with respect to the total weight of the composition. More particularly, the cellulose ether is selected from the group consisting of hydroxypropyl methylcellulose, methyl cellulose, hydroxyethyl cellulose, hydroxypropyl cellulose, carboxymethylcellulose, and combinations thereof.

In another embodiment, optionally in combination with one or more features of the various embodiments described above or below, the cellulose ether is hydroxypropyl methylcellulose, more particularly, in an amount from 0.01 to 5% w/w, more particularly from 0.5 to 2% w/w, with respect to the total weight of the composition. In another embodiment, the hydroxypropyl methylcellulose is present in an amount about 0.01% w/w, about 0.05% w/w, about 0.1% w/w, about 0.2% w/w, about 0.3% w/w, about 0.4% w/w, about 0.5% w/w, about 0.6% w/w, about 0.62% w/w, about 0.7% w/w, about 0.75% w/w, about 0.8% w/w, about 0.9% w/w, about 1% w/w, about 1.1% w/w, about 1.2% w/w, about 1.22% w/w, about 1.3% w/w, about 1.4% w/w about 1.5% w/w, about 1.6% w/w, about 1.62% w/w, about 1.63% w/w, about 1.7% w/w, about 1.8% w/w, about 1.9% w/w, about 2% w/w, about 2.5% w/w, about 3% w/w, about 3.5% w/w, about 4% w/w, about 4.5% w/w, or about 5% w/w, with respect to the total weight of the composition.

In another embodiment, optionally in combination with one or more features of the various embodiments described above or below, the cellulose ether is methyl cellulose (MC), more particularly, in an amount from 0.01 to 5% w/w, more particularly from 0.5 to 2% w/w, with respect to the total weight of the composition. In another embodiment, the methyl cellulose is present in an amount about 0.01% w/w, about 0.05% w/w, about 0.1% w/w, about 0.2% w/w, about 0.3% w/w, about 0.4% w/w, about 0.5% w/w, about 0.6% w/w, about 0.62% w/w, about 0.7% w/w, about 0.75% w/w, about 0.8% w/w, about 0.9% w/w, about 1% w/w, about 1.1% w/w, about 1.2% w/w, about 1.22% w/w, about 1.3% w/w, about 1.4% w/w about 1.5% w/w, about 1.6% w/w, about 1.62% w/w, about 1.63% w/w, about 1.7% w/w, about 1.8% w/w, about 1.9% w/w, about 2% w/w, about 2.5% w/w, about 3% w/w, about 3.5% w/w, about 4% w/w, about 4.5% w/w, or about 5% w/w, with respect to the total weight of the composition.

In another embodiment, optionally in combination with one or more features of the various embodiments described above or below, the poloxamer is selected from the group consisting of poloxamer 407, poloxamer 188, and combinations thereof. More particularly, the poloxamer or poloxamers are present in an amount from 10 to 22% w/w, more particularly from 12 to 18% w/w, with respect to the total weight of the composition. In another embodiment, the poloxamer or poloxamers is present in an amount about 10% w/w, about 11% w/w, about 12% w/w, about 13% w/w, about 14% w/w, about 15% w/w, about 16% w/w, about 16.1% w/w, about 16.2% w/w, about 16.3% w/w, about 16.4% w/w, about 16.5% w/w, about 17% w/w, about 18% w/w, about 19% w/w, about 20% w/w, about 21% w/w, or about 22% w/w, with respect to the total weight of the composition.

In another embodiment, optionally in combination with one or more features of the various embodiments described above or below, the poloxamer is poloxamer 407, more particularly in an amount from 10 to 22% w/w, more particularly from 12 to 18% w/w, with respect to the total weight of the composition. In another embodiment, the poloxamer 407 is present in an amount about 10% w/w, about 11% w/w, about 12% w/w, about 13% w/w, about 14% w/w, about 15% w/w, about 16% w/w, about 16.1% w/w, about 16.2% w/w, about 16.3% w/w, about 16.4% w/w, about 16.5% w/w, about 17% w/w, about 18% w/w, about 19% w/w, about 20% w/w, about 21% w/w, or about 22% w/w, with respect to the total weight of the composition.

In another embodiment, optionally in combination with one or more features of the various embodiments described above or below, the poloxamer is a combination of poloxamer 407, and poloxamer 188. More particularly, the poloxamer 407 is present in an amount from 12 to 20% w/w, more particularly from 14 to 18% w/w with respect to the total weight of the composition, even more particularly in an amount about 14% w/w, 14.5% w/w, about 15% w/w, about 15.5% w/w, about 15.7% w/w, about 15.8% w/w, about 15.9% w/w, about 16% w/w, about 16.1% w/w, about 16.2% w/w, about 16.3% w/w, about 16.4% w/w, about 16.5% w/w, about 17% w/w, about 17.5% w/w, or about 18% w/w, with respect to the total weight of the composition; and the poloxamer 188 is present in an amount from 0.01 to 1% w/w, more particularly from 0.1 to 0.6% w/w, with respect to the total weight of the composition, even more particularly in an amount about 0.18% w/w, about 0.19% w/w, 0.2% w/w, about 0.21% w/w, about 0.22% w/w, about 0.23% w/w, about 0.24% w/w, about 0.25% w/w, about 0.3% w/w, about 0.35% w/w, about 0.4% w/w, about 0.41% w/w, about 0.42% w/w, about 0.43% w/w, about 0.44% w/w, about 0.45% w/w, about 0.5% w/w, about 0.55% w/w, or about 0.6% w/w, with respect to the total weight of the composition.

In another embodiment, optionally in combination with one or more features of the various embodiments described above or below, the poloxamer is a combination of poloxamer 407, and poloxamer 188 wherein the weight ratio between poloxamer 407 and poloxamer 188 is from 20:1 to 90:1, more particularly from 30:1 to 80:1, and even more particularly is about 30:1, about 35:1, about 40:1, about 45:1, about 50:1, about 55:1, about 60:1, about 65:1, about 70:1, about 75:1, about 79:1, or about 80:1.

In another embodiment, optionally in combination with one or more features of the various embodiments described above or below, the one or more adhesive agents comprise:
i) a cellulose ether, particularly in an amount from 0.01 to 5% w/w, more particularly from 0.5 to 2% w/w, with respect to the total weight of the composition, particularly selected from the group consisting of hydroxypropyl methylcellulose, methyl cellulose, carboxymethylcellulose, hydroxyethyl cellulose, hydroxypropyl cellulose, and combinations thereof, more particularly selected from the group consisting of hydroxypropyl methylcellulose and methyl cellulose, and
ii) a poloxamer, which can be a single poloxamer or a combination of poloxamers, particularly poloxamer 407 and/or poloxamer 188, particularly in an amount from 10 to 22% w/w, more particularly from 12 to 18% w/w, with respect to the total weight of the composition.

In another embodiment, optionally in combination with one or more features of the various embodiments described above or below, the one or more adhesive agents comprise a cellulose ether, and a poloxamer, wherein the weight ratio between the poloxamer, and the cellulose ether is from 5:1 to 40:1, more particularly from 5:1 to 30:1, even more particularly is about 5:1, about 10:1, about 15:1, about 20:1, about 25:1, or about 30:1.

In one embodiment, optionally in combination with one or more features of the various embodiments described above or below, the topical composition of the invention comprises two adhesive agents, wherein:
i) one adhesive agent is a cellulose ether, particularly in an amount from 0.01 to 5% w/w, more particularly from 0.5 to 2% w/w, with respect to the total weight of the composition, particularly selected from the group consisting of hydroxypropyl methylcellulose, carboxymethylcellulose, methyl cellulose, hydroxyethyl cellulose, hydroxypropyl cellulose, and combinations thereof, more particularly selected from the group consisting of hydroxypropyl methylcellulose and methyl cellulose, and
ii) the other adhesive agent is a poloxamer, particularly poloxamer 407, particularly in an amount from 10 to 22% w/w, more particularly from 12 to 18% w/w, with respect to the total weight of the composition.

In one embodiment, optionally in combination with one or more features of the various embodiments described above or below, the topical composition of the invention comprises three adhesive agents, wherein:
i) one adhesive agent is a cellulose ether, particularly in an amount from 0.01 to 5% w/w, more particularly from 0.5 to 2% w/w, with respect to the total weight of the composition, particularly selected from the group consisting of hydroxypropyl methylcellulose, methyl cellulose, carboxymethylcellulose, hydroxyethyl cellulose, hydroxypropyl cellulose, and combinations thereof, more particularly selected from the group consisting of hydroxypropyl methylcellulose and methyl cellulose, and
ii) two adhesive agents are poloxamers, particularly poloxamer 407 and poloxamer 188, wherein in particular the first poloxamer, particularly the poloxamer 407, is present in an amount from 12 to 20% w/w, more particularly from 14 to 18% w/w, with respect to the total weight of the composition; and the second poloxamer, particularly poloxamer 188, is present in an amount from 0.01 to 1% w/w, more particularly from 0.1 to 0.6% w/w, with respect to the total weight of the composition.

The composition of the invention is thermoreversible. This means that it is a liquid composition at room temperature, and a gel at body temperature. In particular, the composition of the invention at body temperature forms a thick film, more particularly a film with a thickness from 0.5 to 5 mm, as opposed to a thin film, when applied to the mucosa. This has the advantage that it further improves the physiological healing process of the mucosal lesion.

In one embodiment, optionally in combination with one or more features of the various embodiments described above or below, the adhesion of the topical composition at room temperature is lower than the adhesion of the topical composition at body temperature.

In another embodiment, optionally in combination with one or more features of the various embodiments described above or below, the viscosity of the topical composition at room temperature is lower than the viscosity of the topical composition at body temperature.

In another embodiment, optionally in combination with one or more features of the various embodiments described above or below, the viscosity of the topical composition at body temperature is from 50 to 190 Pa·s, more particularly from 90 to 150 Pa·s. More particularly, in this embodiment, the composition comprises poloxamers.

In another embodiment, optionally in combination with one or more features of the various embodiments described above or below, the topical composition of the invention an aqueous composition comprising water or a buffer. More particularly, the buffer is phosphate-buffered saline (PBS).

PBS is a commonly used water-based salt solution having a pH at about 7.4 which contains disodium hydrogen phosphate, sodium chloride and, in some formulations, potassium chloride and potassium dihydrogen phosphate.

The topical composition comprises a non-absorbable antibiotic in an amount from 0.005 to 0.5% w/w with respect to the total weight of the composition. The term “non-absorbable antibiotic” refers to a compound having antibacterial properties which is poorly or not absorbed from the lumen, i.e., the non-absorbable antibiotic provides activity only locally in the gut and has a negligible systemic absorption. Non-absorbable antibiotics, unlike systemically available antibiotics, maintain a topical action without systemic and side effects and the lack of resistant bacterial strains may allow prolonged and repeated treatments. This definition does not include the compounds that are commonly used as antiseptic, such as chlorhexidine.

In one embodiment, optionally in combination with one or more features of the various embodiments described above or below, the non-absorbable antibiotic is other than chlorhexidine.

In another embodiment, optionally in combination with one or more features of the various embodiments described above or below, the non-absorbable antibiotic is selected from the group consisting of gentamicin, vancomycin, nystatin, neomycin, colistin, kanamycin, polimixin, and rifaximin. More particularly, the non-absorbable antibiotic is rifaximin.

In another embodiment, optionally in combination with one or more features of the various embodiments described above or below, the non-absorbable antibiotic, particularly rifaximin, is present in an amount 0.01 to 0.4% w/w with respect to the total weight of the composition, more particularly in an amount about 0.01% w/w, about 0.016% w/w, about 0.02% w/w, about 0.05% w/w, about 0.75% w/w, about 0.1% w/w, about 0.12% w/w, about 0.15% w/w, about 0.2% w/w, about 0.24% w/w, about 0.25% w/w, about 0.3% w/w, or about 0.4% w/w, with respect to the total weight of the composition.

The topical composition of the invention may also comprise further components, such as for example mineral cofactors, more particularly cofactors for Matrix metalloproteinases (MMPs). As used herein, “cofactor” refers to an agent that activates an enzyme, more particularly an endopeptidase, such as MMP.

Examples of mineral cofactors of the formulation may include zinc compounds, calcium compounds, manganese compounds, and magnesium compounds. Particularly suitable mineral cofactors are zinc cofactors such as zinc oxide, zinc gluconate, zinc amino acid chelates or mixtures thereof.

In one embodiment, optionally in combination with one or more features of the various embodiments described above or below, the topical composition comprises a mineral cofactor, more particularly a mineral cofactors for Matrix metalloproteinases (MMPs), more particularly a zinc cofactor, even more particularly zinc oxide. The cofactor may be present in the composition in an amount from 4 to 10 wt % by weight, more particularly 6 to 8% by weight, with respect to the total weight of the topical composition.

In one embodiment, optionally in combination with one or more features of the various embodiments described above or below, the invention refers to a topical composition, particularly an aqueous topical composition, which comprises or consists of:
a) a hyaluronic acid or a pharmaceutically or veterinary acceptable salt thereof, particularly a hyaluronic acid salt in an amount from 0.25 to 1.5% w/w, more particularly from 0.55 to 1.25% w/w;
b) one or more adhesive agents, particularly i) a first adhesive agent, more particularly a cellulose ether, more particularly hydroxypropyl methylcellulose or methyl cellulose, particularly in an amount from 0.01 to 5% w/w; and ii) a poloxamer, which can be a single poloxamer or a combination of poloxamers, particularly poloxamer 407 and/or poloxamer 188, particularly in an amount from 10 to 22% w/w with respect to the total weight of the composition;
c) a non-absorbable antibiotic, particularly rifaximin, in an amount from 0.005 to 0.5% w/w, more particularly 0.01 to 0.4% w/w;
   wherein the % w/w percentages are expressed with respect to the total weight of the composition, provided that the sum of the amounts of the components is equal to or less than 100%.

The topical composition of the invention is biodegradable. This means that it is bioresorbed or degraded or broken down into components that are well tolerated by the body of the patient. Thus, there is no need to remove the composition of the invention once applied to the body.

The composition of the invention may be prepared by mixing their components, particularly in water or a buffer, and stirring until achieving the complete dissolution of the components.

The composition of the invention is to be administered in a therapeutically effective amount sufficient to cover the affected mucosal area.

A mentioned above it also forms part of the injection device comprising the topical composition as previously defined, and a kit comprising this injection device and a delivery device that is suitable to be coupled to the injection device.

The injection device may be any device appropriate for comprising the composition of the invention that is suitable to be coupled or connected to the delivery device. Non-limiting examples of injection devices include syringes or jet injectors.

The delivery device can be any tubular device having a lumen that is suitable to be coupled or connected to the injection device and is capable to deliver the composition of the invention to its action site. Non-limiting examples of delivery devices include catheters.

In one embodiment, optionally in combination with one or more features of the various embodiments described above or below, the delivery device has a smaller diameter than the working channel diameter of the endoscope.

For example, when the topical composition is to be administered to the gastrointestinal mucosal after therapeutic endoscopy, it can be applied by using an appropriate delivery device or system such as a catheter which can be introduced via an endoscope. Thus, for this therapeutic application, the delivery device has a smaller diameter than the working channel diameter of the endoscope.

The endoscope can be the same endoscope used to carry out the therapeutic endoscopy.

Generally, gastrointestinal endoscopes have working channel diameter in the range of from 2.8 to 4.2 mm and a length of about 190 cm. In one embodiment, the delivery device has an external diameter equal to or lower than 4.2 mm, more particularly, equal to or lower than 3.7 mm, and a length equal to or higher than 160 cm, more particularly higher than 180 cm. For example, the length of the delivery device may be about 190 cm.

The skilled in the art will know the injection device to be chosen depending on the delivery device to be used so that the composition may be administered by using an adequate force.

For example, in one embodiment, optionally in combination with one or more features of the various embodiments described above or below, the invention relates to a delivery device, particularly a catheter, comprising the topical composition as previously defined, wherein the delivery device has an internal diameter in the range from 1.4 to 2.2 mm, and more particularly from 1.6 to 1.8 mm, and the injector device is a syringe. In this case, the composition may be administered by applying a force from 10 to 70 N. This force, referred to as dynamic glide force (DGF), is the force required to sustain the movement of the plunger to expel the contents of the syringe may be measured by means of a dynamometer.

In another embodiment, optionally in combination with one or more features of the various embodiments described above or below, the invention relates to a delivery device, particularly a catheter, comprising the topical composition as previously defined, wherein the delivery device has a diameter in the range of 0.6-0.8 mm, and the injector device is a jet injector.

The expression “therapeutically effective amount” as used herein, refers to the amount of the composition of the invention that, when administered, is sufficient to prevent development of, or alleviate to some extent, one or more of the symptoms of the disease which is addressed. The specific dose of the topical composition to obtain a therapeutic benefit may vary depending on the particular circumstances of the case. A typical amount to be sprayed is from 0.25 to 2 g per cm² of mucosal lesion.

For the purposes of the invention, each component of the topical composition as defined above must be pharmaceutically or veterinary acceptable in the sense of being compatible with the other ingredients of the composition. It must also be suitable for use in contact with the tissue or organ of humans and animals without excessive toxicity, irritation, allergic response, immunogenicity or other problems or complications commensurate with a reasonable benefit/risk ratio.

As mentioned above, the topical composition as defined above may be used in the treatment of mucosal lesions and/or for the prevention of complications derived from mucosal lesions.

The composition of the invention is suitable for being administered to a mucosa, and/or suitable for healing mucosal lesions, in particular, mucosal lesions induced by thermal injury, and more particularly, thermal injury associated or caused by therapeutic endoscopy.

In one embodiment, optionally in combination with one or more features of the various embodiments described above or below, the invention relates to the topical composition as defined above for use in the treatment and/or prevention of mucosal lesions induced by thermal injury, more particularly, thermal injury associated or caused by therapeutic endoscopy.

As used herein, thermal injury refers to an injury caused by either extreme cold or heat which alters or damages the tissue, chemical or electrical burn which alters or damages the tissue, or chemical or electrical trauma which alters or damages the tissue.

Generally, the therapeutic endoscopy includes polypectomy, Endoscopic Mucosal Resection (EMR), and Endoscopic Submucosal Dissection (ESD).

In another embodiment, optionally in combination with one or more features of the various embodiments described above or below, the invention relates to a topical composition as defined above for use in the prevention of postpolypectomy syndrome.

In another embodiment, optionally in combination with one or more features of the various embodiments described above or below, the invention relates to a topical composition as defined above for use in the treatment and/or prevention of mucosal lesions selected from the group consisting of gastrointestinal tract mucosal lesions, vaginal mucosal lesions and oral lesions.

In another embodiment, optionally in combination with one or more features of the various embodiments described above or below, the invention relates to the topical composition as defined above for use in the treatment and/or prevention of mucosal lesions secondary to radiotherapy (actinic proctitis).

In another embodiment, optionally in combination with one or more features of the various embodiments described above or below, the invention relates to the topical composition as defined above for use in the treatment and/or prevention of mucosal lesions which are mucosal perforations, more particularly gastrointestinal perforations. More particularly, the invention relates to the topical composition as defined above may be used as adjuvant therapy to mechanical treatments in gastrointestinal perforations, more particularly, gastrointestinal perforations secondary to endoscopy.

As mentioned above, the topical composition as defined above may also be used as sealant treatment in surgical anastomoses and leaks or fistulas in the gastrointestinal tract, in particular to prevent leaks after surgery.

As mentioned above, the compositions of the invention show a higher mucosal healing rate and a higher physiological healing, while reducing at the same time the fibrotic healing in comparison to a composition consisting only of hyaluronic acid.

As used herein, the term “physiological healing” refers to the restoration of damaged living tissue, organs and biological system to normal function. It is the process by which the cells in the body regenerate and repair to reduce the size of a damaged or necrotic area. The term “fibrotic healing” refers to the temporal and progressive deposition of fibrous tissue over the affected tissue during fibrosis. Generally, when fibrotic healing occurs, a scar is formed which may be cumbersome and vulnerable to repeated trauma.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples and drawings are provided by way of illustration, and they are not intended to be limiting of the present invention. Furthermore, the present invention covers all possible combinations of particular and preferred embodiments described herein.

### Examples

### 1. Materials

Hyaluronic acid (HA) sodium salt: Also known as poly (beta-glucuronic acid (1-3)-beta-N-acetylglucosamine-(1-4). Average Molecular Weight (0.01-0.02 MDa or 0.5-0.75 MDa) Hydroxymethylpropyl cellulose or hypromellose (HPMC). Also known as BonuCel D15H with metoxy substitution between 28-30%.

Methylcellulose (MC): Also known as MethoCel A®, Methylcellulose A, Methylcellulose ether. Approximate molecular weight: 14000 g/mol; Cellulose with methoxy substitution between 27.5-31.5%.

Poloxamer 407: Also known as Kolliphor® P407 Poly (ethylene glycol)-block-poly(propylene glycol)-block-poly(ethylene glycol); Pluronic®F127. Oxyethylene content 71.5-74.9%. Average molecular weight: 9849-14600 g/mol.

Poloxamer 188: Also known as Kolliphor®P188 Poly (ethylene glycol)-block-poly(propylene glycol)-block-poly(ethylene glycol); Pluronic®F68. Oxyethylene content 79.9-83.7%; Average molecular weight 7680-9510 g/mol.

Polyvinyl alcohol (PVA): 87-89% hydrolyzed. Average molecular weight 88000-97000 g/mol.

Alginate: sodium alginate, also known as algin. Granulometry 30-150 mesh. Viscosity (1%) (R2, 20 rpm)>300 cps.

CaCl₂): calcium chloride. Molecular weight 147.02 Da as calcium chloride dihydrate CaCl₂) 2H₂O.

Carboxymethyl cellulose (CMC): carboxymethylcellulose, sodium salt, high viscosity. 2%, H₂O, 25° C.): 800-3100 mPas.

Hydroxyethyl cellulose (HEC): partially O-(2-hydroxyethylated) cellulose. Viscosity (2%, 25° C.): 1500-2500 mPa·s

Dextran: Dextran from Leuconostoc spp. Mr ˜40000 g/mol (specification 35000-47000 g/mol)

Sodium silicate (Na₂SiO₃): sodium silicate solution 13.4-14.4% (Titration by HCl, % NaOH); Gravimetric analysis 12.0-13.0% (% Si)

Polyvinyl acetate: Poly(vinyl acetate)-average Mw ˜100000 by GPC (beads)

Starch: starch soluble GR for analysis ISO; pH value (2%; water) 6.0-7.5; reducing matter as maltose (max. 0.7%); Suitability as enzyme substrate (for amylases) passes test.

Rifaximin (RFX): Also known as Rifacol; 4-Deoxy-4′-methylpyrido[1′,2′-1,2]imidazo[5,4-c]rifamycin SV; Molecular Weight: C₄₃H₅₁N₃O₁₁; Molecular Weight: 785.88 g/mol

### 2. Protocol for the Preparation of the Compositions

### On-Bench:

### Protocol 2A

Rifaximin (different amounts, see tables below) was added to PBS (Phosphate-buffered saline, pH ˜7.4, 100 mL) in a 250 mL glass beaker and the mixture was stirred at 300 rpm at 50° C. Then, if present, hyaluronic acid sodium salt (1 g) was added and stirred at 300 rpm until its complete dissolution. Then, the cellulose (2 g) was added and stirred again to 300 rpm until its complete dissolution. The mixture was placed on a closed-container and cooled at 4° C. Finally, a mixture of poloxamers (Poloxamer P407 and Poloxamer P188, 20 g) or other adhesive agent was added to the mixture at several portions and vortexed several times to disperse the solids. The container was placed on a roller and stirred at 70 rpm for two days. Then, the following compositions were obtained: The amounts of PBS, hyaluronic acid sodium salt, cellulose derivative as well as adhesive agent can be scaled according to the needs of the process.

TABLE 1

| | Amounts | | | | |
|---|---|---|---|---|---|
| Component | Example 1 | Example 2 | Example 3 | Example 4 | Example 5 |
| HA sodium salt | 1 g | 1 g | — | — | — |
| (MW: 0.01-0.02 MDa) | (0.81% w/w) | (0.81% w/w) | | | |
| HA sodium salt | — | — | 1 g | 1 g | 1 g |
| (MW: 0.5-0.75 MDa) | | | (0.81% w/w) | (0.81% w/w) | (0.81% w/w) |
| HPMC | 2 g | 2 g | — | — | — |
| | (1.63% w/w) | (1.63% w/w) | | | |
| MC | — | — | 2 g | 2 g | 2 g |
| | | | (1.62% w/w) | (1.62% w/w) | (1.62% w/w) |
| Poloxamer P407 | 19.75 g | 19.75 g | 19.5 g | 19.5 g | 19.5 g |
| | (16.05% w/w) | (16.05% w/w) | (15.83% w/w) | (15.82% w/w) | (15.85% w/w) |
| Poloxamer P188 | 0.25 g | 0.25 g | 0.5 g | 0.5 g | 0.5 g |
| | (0.20% w/w) | (0.20% w/w) | (0.41%, w/w) | (0.41% w/w) | (0.40% w/w) |
| Rifaximin (RFX) | 0.02 g | 0.02 g | 0.15 g | 0.3 g | 0.5 g |
| | (0.016%) | (0.016%) | (0.12% w/w) | (0.24% w/w) | (0.40% w/w) |
| PBS | 100 g | 100 g | 100 g | 100 g | 100 g |
| | (81.29%) | (81.29%) | (81.20% w/w) | (81.10% w/w) | (80.97%) |
| Total | 123.02 g | 123.02 g | 123.15 g | 123.30 g | 123.50 g |
| | (100% w/w) | (100% w/w) | (100% w/w) | (100% w/w) | (100% w/w) |

TABLE 2

| | Amounts | | | | |
|---|---|---|---|---|---|
| Component | Example 6 | Example 7 | Example 8 | Example 9 | Example 10 |
| HA sodium salt | 1 g | 1 g | — | — | 1 g |
| (MW: 0.01-0.02 MDa) | (0.82% w/w) | (0.82% w/w) | | | (0.81% w/w) |
| HA sodium salt | — | — | 1 g | 1 g | — |
| (MW: 0.5-0.75 M Da) | | | (0.81% w/w) | (0.81% w/w) | |
| HPMC | 1.50 g | 0.75 g | — | — | 2 g |
| | (1.22% w/w) | (0.62% w/w) | | | (1.63% w/w) |
| MC | — | — | 2 g | 2 g | — |
| | | | (1.62% w/w) | (1.62% w/w) | |
| Poloxamer P407 | 20 g | 19.75 g | 20 g | 20 g | 20 g |
| | (16.32% w/w) | (16.22% w/w) | (16.24% w/w) | (16.24% w/w) | (16.26% w/w) |
| Poloxamer P188 | — | 0.25 g | — | — | — |
| | | (0.21% w/w) | | | |
| Rifaximin (RFX) | 0.02 g | 0.02 g | 0.15 g | 0.15 g | 0.02 g |
| | (0.016% w/w) | (0.016% w/w) | (0.112% w/w) | (0.112% w/w) | (0.016% w/w) |
| PBS | 100 g | 100 g | 100 g | 100 g | 100 g |
| | (81.62% w/w) | (82.12% w/w) | (81.20% w/w) | (81.20% w/w) | (81.29% w/w) |
| Total | 122.52 g | 121.77 g | 123.15 g | 123.15 g | 123.02 g |
| | (100% w/w) | (100% w/w) | (100% w/w) | (100% w/w) | (100% w/w) |

TABLE 3

| | Amounts | | | |
|---|---|---|---|---|
| | Comparative | Comparative | Comparative | Comparative |
| Component | example 11 | example 12 | example 13 | example 14 |
| HA sodium salt | — | — | — | 1 g |
| (MW: 0.01-0.02 MDa) | | | | (0.81% w/w) |
| HA sodium salt | 1 g | 1 g | 1 g | — |
| (MW: 0.5-0.75 M Da) | (0.81% w/w) | (0.80% w/w) | (0.80% w/w) | |
| HPMC | — | — | — | 2 g |
| | | | | (1.63% w/w) |
| MC | 2 g | 2 g | 2 g | — |
| | (1.63% w/w) | (1.61% w/w) | (1.61% w/w) | |
| Poloxamer P407 | 19.5 g | 19.5 g | 20 g | 20 g |
| | (15.85% w/w) | (15.66% w/w) | (16.06% w/w) | (16.26% w/w) |
| Poloxamer P188 | 0.5 g | 0.5 g | — | — |
| | (0.41% w/w) | (0.40% w/w) | | |
| Rifaximin (RFX) | 0 | 1.5 g | 1.5 g | — |
| | | (1.20% w/w) | (1.20% w/w) | |
| PBS | 100 g | 100 g | 100 g | 100 g |
| | (81.30% w/w) | (80.32% w/w) | (80.32% w/w) | (81.30% w/w) |
| Total | 123 g | 124.5 g | 124.5 g | 123 g |
| | (100% w/w) | (100% w/w) | (100% w/w) | (100% w/w) |

TABLE 4

Amounts

| | Comparative | Comparative |
|---|---|---|
| Component | example 15 | example 16 |
| HA sodium salt | — | — |
| (MW: 0.01-0.02 MDa) | | |
| HA sodium salt | — | — |
| (MW: 0.5-0.75 M Da) | | |
| HPMC | 2 g | 2 g |
| | (1.64% w/w) | (1.64% w/w) |
| MC | — | — |
| Poloxamer P407 | 20 g | 20 g |
| | (16.39% w/w) | (16.39% w/w) |
| Poloxamer P188 | — | — |
| Rifaximin (RFX) | 0.02 g | — |
| | (0.016% w/w) | |
| PBS | 100 g | 100 g |
| | (81.95% w/w) | (81.97% w/w) |
| Total | 122.02 g | 122 g |
| | (100% w/w) | (100% w/w) |

Following the same protocol above but substituting the mixture of poloxamers by the polymers indicated in the table below, further compositions according to the invention were obtained:

TABLE 5

| | Amounts | | | |
|---|---|---|---|---|
| Component | Example 17 | Example 18 | Example 19 | Example 20 |
| HA sodium salt | 0.48 g | 0.46 g | 0.48 g | 0.48 g |
| (MW: 0.01-0.02 MDa) | (0.81% w/w) | (0.80% w/w) | (0.81% (w/w) | (0.81% w/w) |
| HPMC | 0.96 g | 0.93 g | 0.97 g | 0.97 g |
| | (1.62% w/w) | (1.60% w/w) | (1.62% w/w) | (1.62% w/w) |
| Adhesive agent | PVA | Alginate + CaCl₂ | CMC | HEC |
| | 10.00 g | 10.13 g | 10.00 g | 10.00 g |
| | (16.76% w/w) | (17.45% w/w) | (16.69% w/w) | (16.69% w/w) |
| Rifaximin (RFX) | 0.010 g | 0.0093 g | 0.010 g | 0.010 g |
| | (0.016% w/w) | (0.016% w/w) | (0.016% w/w) | (0.016% w/w) |
| PBS | 48.23 g | 46.55 g | 48.43 g | 48.34 g |
| | (80.80% w/w) | (80.13% w/w) | (80.86% w/w) | (80.84% w/w) |
| Total | 59.68 g | 58.08 g | 59.89 g | 59.80 g |
| | (100% w/w) | (100% w/w) | (100% w/w) | (100% w/w) |

TABLE 6

| | Amounts | | | |
|---|---|---|---|---|
| Component | Example 21 | Example 22 | Example 23 | Example 24 |
| HA sodium salt | 0.24 g | 0.47 g | 0.24 g | 0.24 g |
| (MW: 0.01-0.02 MDa) | (0.81% w/w) | (0.81% w/w) | (0.81% w/w) | (0.81% w/w) |
| HPMC | 0.48 g | 0.95 g | 0.47 g | 0.47 g |
| | (1.62% w/w) | (1.62% w/w) | (1.61% w/w) | (1.61% w/w) |
| Adhesive agent | Dextran | Sodium silicate | Polyvinyl acetate | Starch |
| | 5.00 g | 10.00 g | 4.99 g | 5.01 g |
| | (16.72% w/w) | (17.00% w/w) | (17.01% w/w) | (17.05% w/w) |
| Rifaximin (RFX) | 0.0049 g | 0.0095 | 0.0047 g | 0.0047 g |
| | (0.016% w/w) | (0.016% w/w) | (0.016% w/w) | (0.016% w/w) |
| PBS | 24.20 g | 47.42 g | 23.65 g | 23.66 g |
| | (80.83% w/w) | (80.55% w/w) | (80.55% w/w) | (80.51% w/w) |
| Total | 29.92 g | 58.85 g | 29.35 g | 29.38 g |
| | (100% w/w) | (100% w/w) | (100% w/w) | (100% w/w) |

### 3. Hydrogel Characterization

### Rheological Assay

A rheological study was performed in a rheometer Discovery HR-1 TA instruments. In first place, the sample was conditioned at 21° C. for 600 s. Then, the viscosity was measured at shear rate 1 s⁻¹ at 21° C. for 120 s. After that, the viscosity was measured at shear rate 1 s⁻¹ at 37° C. for 360 s. Viscosity data are plotted against time and the time of gelling was also determined. Table 7 shows the viscosity data at 37° C. obtained for the different formulations (the letters a and b for each example represent different samples of the same formulation). Viscosities at 37° C. were determined at 150 seconds (half of the experiment) and 360 seconds (end of the experiment).

TABLE 7

Viscosity (Pa · s)

| | | At 37° C. | At 37° C. |
|---|---|---|---|
| Formulation | At 21° C. | (150 s) | (360 s) |
| Example 1a | 0.234 | 121.316 | 122.380 |
| Example 1b | 0.216 | 100.621 | 101.538 |
| Example 2a | 0.300 | 151.213 | 158.013 |
| Example 2b | 0.286 | 125.968 | 115.707 |
| Example 3a | 3.107 | 67.170 | 89.297 |
| Example 3b | 1.460 | 78.523 | 104.586 |
| Example 4a | 7.743 | 92.595 | 117.817 |
| Example 4b | 5.333 | 102.622 | 114.333 |
| Comparative example 12 | 1.890 | 78.593 | 109.000 |

### Viscosity at 21° C. And Injectability

Samples containing hyaluronic acid of 0.01-0.02 MDa (examples 1 and 2) showed a viscosity at 21° C. lower than those containing HA of 0.5-0.75 MDa (examples 3 and 4). This is related to the fluidity of the formulation and injectability, and thus usability, being particularly good values those that are below 1 Pa·s. Injectability, and fluidity were tested using a texturometer and determining the injection force to be applied to a syringe containing the formulation connected to a catheter of a certain length. The results are shown in the table below.

TABLE 8

| Formulation | DGF (N) | Fmax (N) |
|---|---|---|
| Example 1 (catheter lenght: 1.50 m) | 10.5 | 12.5 |
| Example 1 (catheter lenght: 1.80 m) | 26.3 | 42.7 |
| Example 3 (catheter lenght: 1.50 m) | 40.7 | 60.1 |

DGF: dynamic glide force: force required to sustain the movement of the plunger to expel the content of the syringe, Fmax: maximum force

Table 8 shows that the forces to inject formulations with HA of lower molecular weight (Example 1) were lower than those applied with HA of higher molecular weight (Example 3), even when the catheter was longer.

### Viscosity at 37° C.:

The optimum viscosity range at 37° C. (360 s) is calculated for values above 85 and 200 Pa·s. Table 7 shows the viscosities at 37° C. of the formulations containing different RFX percentages and different HA. All the formulations showed similar values of viscosity within the range of the specification, which means that all of them formed a gel at 37° C. When the viscosity was determined at halfway through the experiment (150 s), the viscosities of the samples of Example 1 and Example 2 were similar to the ones obtained at the end of the experiment (360 s). Formulations of Example 3 and Example 4 reached the maximum viscosity at the end of the experiment (360 s).

### Gelling Time:

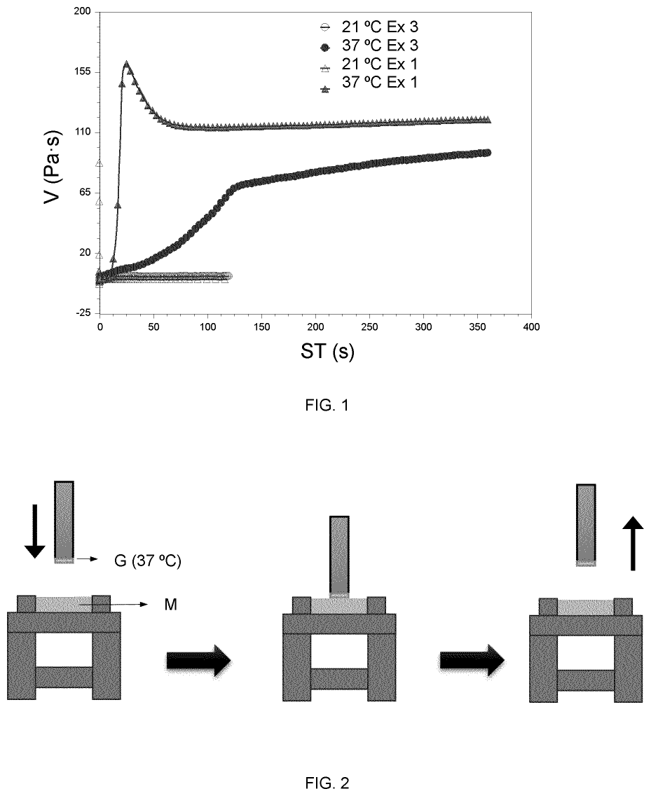

The difference of viscosities measured at 150 s and 360 s observed in FIG. 1 is related to the gelling time. The gelling time is defined as the time needed to reach the maximum viscosity from a temperature of 21° C. to a temperature of 37° C. This time is calculated from the rheology curves. The most favorable gelling time is estimated below 60 seconds. This time is defined for the formulation keeps the fluidity along the catheter and gels as soon as it leaves the catheter remaining adhered to the intestinal mucosa. As can be seen in the table, the fastest times are those that correspond to formulations with Examples 1 and 2:

TABLE 9

| Formulation | Gelling time (s) |
|---|---|
| Example 1a | 25 |
| Example 1b | 28 |
| Example 2a | 21 |
| Example 2b | 20-30 |
| Example 3a | 109 |
| Example 3b | 115 |
| Example 4a | 115 |
| Example 4b | 94 |
| Comparative example 12 | 129 |

### Adhesivity Assay

To verify that the product is adhered to the mucosa, formulations with different concentration of HMPC were tested in an adhesive test, following the method shown in FIG. 2. 2 mL of mucin-PBS solution was placed on top of the cylinder space located on bottom of the equipment. The platform was inside a bathroom at 37° C. The gel sample was deposited on a metal cylinder and incubated at 37° C. for 2 minutes for the gelification of the sample. Once the cylinder is connected to the equipment, the experiment was initiated with the slow movement of the cylinder in the mucin solution at a rate of 0.5 mm/s. When the cylinder submitted to a force of 0.05 N, it stopped inside the mucin solution and remained for 30 s. The cylinder then retracted at a speed of 1 mm/s. The area of the Y-axis (force in N) and the X axis (time in seconds) gave it the adhesiveness in N/s. The maximum force and time needed for retraction is this area. After each trial, the mucin solution was renewed. Table 10 shows the data obtained from the adhesion forces:

TABLE 10

| | | Viscosity |
|---|---|---|
| Formulation | F (mN/s) | (Pa · s) |
| Example 1a | 28.5 | 96.476 |
| Example 6b | 30 | 105.408 |
| Example 17 | 7.7 | 24.903 |
| Example 18 | 6.6 | 2654.370 |
| Example 19 | 10.1 | 1260.790 |
| Example 20 | 8.5 | 4622.075 |
| Example 21 | 13.0 | 0.267 |
| Example 22 | 8.9 | 0.047 |
| Example 23 | 16.2 | — |
| Example 24 | 15.6 | — |

Formulations of Examples 1 and 6 showed the adhesion forces of 28.5 and 30 mN/s respectively. Regarding the formulations of examples 17 to 24, they also showed adhesive properties but with a rheological behaviour with higher (examples 18, 19, 20) or lower viscosities (examples 17, 21, 22) than the poloxamer derived formulations, which shows the versatility of the formulations.

### 4. Evaluation of the Susceptibility of the Formulations of the Invention to Attack Escherichia coli

The test consisted of determining whether formulations with different amounts of RFX were susceptible to attack E. coli and E. faecalis, independently.

### Experiment

250 μL of each formulation was dispensed on glass discs (12 mm diameter). The test was performed with three analytical replicates for each bacterium and test condition. In total it was worked with 48 glass discs on which the control formulation (0% RFX) and the test formulations were gelled. The 48 discs with the formulations were incubated in an oven at 37° C. for 60 minutes to ensure that they gel correctly. Then, 50 μL of a known amount of E. coli (1-5×10⁸ cfu/mL) were added to gelled formulations; bacterial inocula were prepared in liquid medium containing only mineral salts. After that, the discs with the formulation were incubated at 37° C. for 36 hours. The number of viable cells was determined using a serial dilution method and plate seeding of discs with the formulations.

The quantification of viable bacterial cells after incubation of formulation with 50 μl of bacterial inoculum at 37° C. for 36 h is shown in the table below:

TABLE 11

| | E. coli | | | | |
|---|---|---|---|---|---|
| | | | | | Bacteria |
| Formulation | REPLICA 1 | REPLICA 2 | REPLICA 3 | Mean | Reduction % |
| Example 3 | 2.73E+05 | 2.52E+05 | 2.68E+05 | 2.64E+05 | 88.75 |
| Example 5 | 1.96E+05 | 1.48E+05 | 1.74E+05 | 1.73E+05 | 92.65 |
| Comparative | 2.57E+06 | 1.97E+06 | 2.51E+06 | 2.35E+06 | *** |
| example 11 | | | | | |
| Comparative | 2.51E+03 | 8.38E+02 | 1.00E+03 | 1.45E+03 | 99.94 |
| example 12 | | | | | |

A reduction of E. coli was observed, with respect to the control (no RFX, comparative example 11), after incubation at 37° C. for 36 h of the bacterium with the formulations at the different concentrations of RFX (0.12%, 0.40% and 1.20% w/w), being respectively the reductions of 88.75%, 92.65% and 99.94%.

### 5. Endoscopic Study in Rats (Study 1 and 2)

The aim of these experiments was to study the effect of the formulations of the invention. Twelve male Harlan rats (270-300 g) were used in the study 1 and 2 respectively. Rats were acclimatized for a minimum of 7 days preoperatively. Rats were kept at constant room temperature (20-22° C.) with a relative humidity (27-31% with aeration under an alternating 12 h cycle of fluorescent light and darkness. The rats were housed individually in polycarbonate box cages with free access to water and food (Teklad LM-485 rodent diet). Rats suffered minimal pain and distress because of the use of anaesthesia. The protocol was approved by the Institutional Animal Care and Use Committee of Hospital Universitari Germans Trials|Pujol. The animals had free access to water, but food was withdrawn 8 h before the initiation of bowel preparation. A rectal enema with saline solutions was performed immediately before colonoscopy.

Rats were anesthetized by 1.5 mL/min of isofluorane in O₂ and were subjected to thermal damage using an electric scalpel at 40 W for 2 seconds. By using a “coagrasper” clamp 2 burns were made in the colon distal to each.

Formulations were administered without mixing with contrast agents. They were applied over mucosal lesions as a coverage shield, with a catheter through the endoscope channel, positioning the tip of the catheter over the ulcer. An amount of 1-1.5 mL was applied in each animal, approximately 0.5 mL per cm² of mucosal lesion. The colonoscopy was performed with the Olympus Video bronchoscope EVIS EXERA II (BF-1T180) endoscope with an outer diameter of 4.9 mm and a working channel of 2.8 mm. Air may be applied for insufflation during endoscopy. Images were obtained through the endoscope in time at different times, focusing on the areas where the bedsores were located. After 7 days, the animals were slaughtered, and samples were taken to evaluate the mucosal healing. Mucosal healing and regeneration were evaluated as the reduction of the mean ulcerated area determined by using Image J software.

Study 1:3 groups of rats were treated with 3 different formulations. The results are shown in the table 12 below:

TABLE 12

| | | Area | | % reduction |
|---|---|---|---|---|
| Group | Example | (cm²) | SD | ulcerated area |
| G1 | Example 8 | 0.127 | 0.067 | 54.4 |
| G2 | Example 9 | 0.126 | 0.052 | 54.6 |
| G3 | Comparative Example 13 | 0.096 | 0.041 | 65.3 |

Mucosal healing was observed in all groups. A reduction on the ulcer area was observed. In groups G1 and G2 the area was reduced 55% respect to the initial value. In G3 (comparative example) containing 1.20% (w/w) RFX (10-fold with respect to G1 and G2), the reduction effect was only improved by 10%.

Study 2:2 groups of rats were treated with 4 different formulations. The results are shown in the table 13 below:

TABLE 13

| | | Area | | % reduction |
|---|---|---|---|---|
| Group | Example | (cm²) | SD | ulcerated area |
| G1 | Example 10 | 0.046 | 0.047 | 88.8 |
| G2 | Comp. Example 15 | 0.415 | 0.290 | 0 |

The G1 formulation with 0.81% w/w HA 0.01-0.02 MDa and 0.016% w/w RFX achieved a reduction of ulcerated area up to 88.8%.

### 6. Stability Study

The objective of the study was to have data on the stability of the product under real storage conditions (5±3° C.). For this purpose, the study was carried out in real time, under both storage conditions. An accelerated study at 40° C. was not proposed since the product gels at 40° C. and no signs of degradation could be observed under these conditions. The samples were stored inside the refrigerator AR1000CL. Six syringes (3 rheology+3 HPLC/pH/osmolarity) from each batch were stored at 5±3° C. The results (T0=start, T1=1 month; t=2 months; t=3 months; t=4 months) are shown in the table below:

TABLE 14

Viscosity (Pa · S)

| Formulation | t0 | t1 | t2 | t3 | t4 |
|---|---|---|---|---|---|
| Example 1a | 121.32 | — | 104.01 | — | 94.30 |
| Example 1b | 100.62 | — | 90.74 | — | 91.0 |
| Comparative | 46.63 | 31.180 | — | 49.77 | — |
| example 12 | | | | | |

The formulation with 1.20% w/w RFX (comparative example 12) showed very low and inconsistent viscosity values (viscosity at t1 decreased with respect to t0 but increased at time 3), indicating that the formulation was not stable due to a lack of homogeneity, probably because the RFX precipitates. On the other hand, in the new formulations with 0.016% (w/w) the trend in the viscosity values was maintained over time. In general, formulations with more RFX tend to settle over time, appearing a pellet corresponding to precipitated RFX. This is reflected in the RFX recoveries in the HPLC assay:

TABLE 15

| Formulation | t0 | t1 | T2 | T4 |
|---|---|---|---|---|
| Example 1a | 84.85 | 87 | 106.5 | 94.2 |
| Example 1b | 89.9 | 81.85 | — | 94.0 |
| Comparative | 82 | 58.6 | — | 101.4 |
| example 12 | | | | |

Formulations with 1.20% w/w RFX lose 20% of RFX at t1 and recover 100% at t4. This inconsistency results from the inhomogeneity and precipitation of the sample. On the other hand, formulations with 0.016% w/w RFX maintained the % of RFX over time.

### CITATION LIST

WO2016135219

For reasons of completeness, various aspects of the invention are set out in the following numbered clauses:

Clause 1. A topical composition comprising:
a) from 0.25 to 1.5% w/w of a hyaluronic acid or a pharmaceutically or veterinary acceptable salt thereof,
b) from 0.01 to 25% w/w of one or more adhesive agents, and
c) from 0.005 to 0.5% w/w of a non-absorbable antibiotic;
wherein the % w/w percentages are expressed with respect to the total weight of the composition, provided that the sum of the amounts of the components is equal to or less than 100%.

Clause 2. The topical composition according to clause 1, wherein the hyaluronic acid or the salt thereof is present in an amount from 0.55 to 1.25% w/w with respect to the total weight of the composition.

Clause 3. The topical composition according to any of the clauses 1-2, wherein the hyaluronic acid or the salt thereof has an average molecular weight from 5,000 to 1,000,000 Daltons.

Clause 4. The topical composition according to any of the clauses 1-3, wherein the hyaluronic acid or the salt thereof is hyaluronate sodium.

Clause 5. The topical composition according to any of the clauses 1-4, wherein the adhesive agents comprise a cellulose ether and a poloxamer.

Clause 6. The topical composition according to clause 5, wherein the cellulose ether is present in an amount from 0.01 to 5% w/w with respect to the total weight of the composition, and the poloxamer is present in an amount from 10 to 22% w/w with respect to the total weight of the composition.

Clause 7. The topical composition according to any of the clauses 5-6, wherein the cellulose ether is selected from the group consisting of hydroxypropyl methylcellulose, methylcellulose, carboxymethylcellulose, and combinations thereof.

Clause 8. The topical composition according to any of the clauses 5-7, wherein the poloxamer is selected from poloxamer 407, poloxamer 188, and combinations thereof.

Clause 9. The topical composition according to any of the clauses 1-8, wherein the non-absorbable antibiotic is present in an amount from 0.01 to 0.4% w/w with respect to the total weight of the composition.

Clause 10. The topical composition according to any of the clauses 1-9, wherein the non-absorbable antibiotic is rifaximin.

Clause 11. The topical composition according to any of the clauses 1-10, which is an aqueous composition comprising water or a buffered solution.

Clause 12. The topical composition according to any of the clauses 1-11, which is an injectable composition at room temperature.

Clause 13. An injection device comprising the topical composition as defined in any of the clauses 1-12.

Clause 14. A kit comprising the injection device as defined in clause 13, and a delivery device suitable to be coupled to the injection device.

Clause 15. A topical composition as defined in any of the clauses 1-11, for use in the treatment and/or prevention of mucosal lesions.

Clause 16. The topical composition for use according to claim 15, wherein the mucosa is gastrointestinal or vaginal mucosa.

Clause 17. A topical composition as defined in any of the clauses 1-12, for use in the prevention of postpolypectomy syndrome, or alternatively, for use as sealant treatment in surgical anastomoses and leaks or fistulas in gastrointestinal tract, or alternatively, for use as adjuvant therapy to mechanical treatments in gastrointestinal perforations.

1. A topical composition suitable for application on gastrointestinal or vaginal mucosa, which comprises:
a) from 0.25 to 1.5% w/w of a hyaluronic acid or a pharmaceutically or veterinary acceptable salt thereof,
b) from 0.01 to 25% w/w of one or more adhesive agents, and
c) from 0.005 to 0.5% w/w of a non-absorbable antibiotic;
wherein the % w/w percentages are expressed with respect to the total weight of the composition, provided that the sum of the amounts of the components is equal to or less than 100%.

2. The topical composition according to claim 1, wherein the hyaluronic acid or the salt thereof is present in an amount from 0.55 to 1.25% w/w with respect to the total weight of the composition.

3. The topical composition according to claim 1, wherein the hyaluronic acid or the salt thereof has an average molecular weight from 5,000 to 1,000,000 Daltons.

4. The topical composition according to claim 1, wherein the hyaluronic acid or the salt thereof is hyaluronate sodium.

5. The topical composition according to claim 1, wherein the adhesive agents comprise a cellulose ether and a poloxamer.

6. The topical composition according to claim 5, wherein the cellulose ether is present in an amount from 0.01 to 5% w/w with respect to the total weight of the composition, and the poloxamer is present in an amount from 10 to 22% w/w with respect to the total weight of the composition.

7. The topical composition according to claim 1, wherein the cellulose ether is selected from the group consisting of hydroxypropyl methylcellulose, methylcellulose, carboxymethylcellulose, and combinations thereof.

8. The topical composition according to claim 1, wherein the poloxamer is selected from poloxamer 407, poloxamer 188, and combinations thereof.

9. The topical composition according to claim 1, wherein the non-absorbable antibiotic is present in an amount from 0.01 to 0.4% w/w with respect to the total weight of the composition.

10. The topical composition according to claim 1, wherein the non-absorbable antibiotic is rifaximin.

11. The topical composition according to claim 1, which is an aqueous composition comprising water or a buffered solution.

12. An injection device comprising the topical composition as defined in claim 1.

13. A kit comprising the injection device as defined in claim 12, and a delivery device suitable to be coupled to the injection device.

14. A method for the treatment of mucosal lesions and/or for the prevention of complications derived from mucosal lesions comprising topically applying a therapeutically effective amount of a topical composition as defined in claim 1, to a subject in need thereof, including a human.

15. A method for the prevention of postpolypectomy syndrome comprising topically applying a therapeutically effective amount of a topical composition as defined in claim 1, to a subject in need thereof, including a human.

16. A method for the sealant treatment in surgical anastomoses and leaks or fistulas in the gastrointestinal tract comprising topically applying a therapeutically effective amount of a topical composition as defined in claim 1, to a subject in need thereof including a human.

17. A method for the adjuvant therapy to mechanical treatments in gastrointestinal perforations comprising topically applying a therapeutically effective amount of a topical composition as defined in claim 1 to a subject in need thereof including a human.

18. The topical composition according to claim 1, wherein
the non-absorbable antibiotic is present in an amount from 0.01 to 0.4% w/w with respect to the total weight of the composition;
the adhesive agents comprise a cellulose ether and a poloxamer, wherein the cellulose ether is present in an amount from 0.01 to 5% w/w with respect to the total weight of the composition, and the poloxamer is present in an amount from 10 to 22% w/w with respect to the total weight of the composition.

19. The topical composition according to claim 1, wherein the adhesive agents comprise a cellulose ether and a poloxamer, and the non-absorbable antibiotic is rifaximin.

20. The topical composition according to claim 19, wherein the hyaluronic acid or the salt thereof has an average molecular weight from 5,000 to 1,000,000 Daltons.
